(19) United States
(12) Patent Application Publication
(10) Pub. No.: US 2026/0224520 A1
(43) Pub. Date: Aug. 6, 2026

(54) ABC TRANSPORTER EXPRESSION PROMOTING AGENT, CHOLESTEROL EXCRETION PROMOTING AGENT, INFLAMMATORY CYTOKINE PRODUCTION INHIBITING AGENT, ANTI-INFLAMMATORY AGENT, AND ARTERIOSCLEROSIS PREVENTING AGENT

(71) Applicant: MEIJI CO., LTD., Tokyo (JP)

(72) Inventors: Michinori MATSUO, Kyoto (JP); Kai NAKAYAMA, Tokyo (JP); Hirofumi GOTO, Tokyo (JP)

(73) Assignee: MEIJI CO., LTD., Tokyo (JP)

(21) Appl. No.: 19/142,849

(22) PCT Filed: Dec. 25, 2023
(86) PCT No.: PCT/JP2023/046529

§ 371 Date: Jun. 24, 2025

(30) Foreign Application Priority Data
Dec. 26, 2022 (JP) 2022-208733

(51) Int. Cl. A61K 31/20; A61K 35/20; A61P 3/06

(52) U.S. Cl. CPC A61K 31/20; A61K 35/20; A61P 3/06

(57) ABSTRACT

An object of the present invention is to provide an ABC transporter expression promoting agent, a cholesterol excretion promoting agent, an inflammatory cytokine production inhibiting agent, an anti-inflammatory agent, and an arteriosclerosis preventing agent, and the present invention provides an ABC transporter expression promoting agent, a cholesterol excretion promoting agent, an inflammatory cytokine production inhibiting agent, an anti-inflammatory agent, and an arteriosclerosis preventive agent each containing at least one selected from 10-hydroxypalmitic acid and 16-hydroxypalmitic acid.

### TECHNICAL FIELD

The present invention relates to an ABC transporter expression promoting agent, a cholesterol excretion promoting agent, an inflammatory cytokine production inhibiting agent, an anti-inflammatory agent, and an arteriosclerosis preventing agent.

### BACKGROUND ART

With the increase in lifestyle-related diseases (such as metabolic syndrome), cardiovascular diseases including arteriosclerotic diseases now account for the second leading cause of death among Japanese people, and the increased cases of cardiovascular diseases have become a medical issue. Accordingly, prevention and treatment of arteriosclerotic diseases are also a pressing social issue.

For example, Patent Literature 1 discloses that 10-hydroxyoctadecanoic acid has the effects of inhibiting accumulation of visceral fat, inhibiting cholesterol absorption in the intestinal tract, reducing the concentration of neutral lipid in the blood, and increasing the concentration of small HDL cholesterol in the blood.

Patent Literature 2 discloses that C₁₈ hydroxy fatty acid, C₁₈ dihydroxy fatty acid, and C₁₈ epoxy fatty acid have the effects of preventing or ameliorating obesity, hyperlipidemia, arteriosclerosis, and coronary artery disease.

Patent Literature 3 discloses that C₁₈ or C₂₀ unsaturated fatty acid having a hydroxyl group or a carbonyl group at 13-position and dihydroxy unsaturated fatty acid having hydroxyl groups at 10- and 13-positions have the effect of improving metabolism.

Patent Literature 4 discloses that C₁₈ unsaturated fatty acid, C₁₈ unsaturated fatty acid having a hydroxyl group or a carbonyl group at 10, 12, or 13-position and C₁₈ dihydroxy unsaturated fatty acid having hydroxyl groups at 10- and 13-positions have an anti-inflammatory effect.

Patent Literature 5 discloses that C₁₈ hydroxy unsaturated fatty acid having a hydroxyl group at 10- or 12-position has the effect of improving metabolism.

### CITATION LIST

### Patent Literatures

Patent Literature 1: Japanese Patent Laid-Open No. 2020-050602
Patent Literature 2: Japanese Patent Laid-Open No. 2009-051732
Patent Literature 3: International Publication No. WO 2015/111700
Patent Literature 4: International Publication No. WO 2015/111701
Patent Literature 5: International Publication No. WO 2014/069227

### SUMMARY OF INVENTION

### Technical Problem

ATP-Binding Cassette (ABC) transporters are membrane proteins involved in the transport of substances through biological membranes using ATP as driving force.

It is known that ABC transporters are present in various types of cells and tissues and important for regulating the intra- and extra-cellular material environments and maintaining the homeostasis of a living body, and that the function or expression level thereof is decreased due to lifestyle change, genetic mutation, and the like. For example, ABC transporters are known to be involved in the cholesterol homeostasis of a living body through lipid transport, and disruption of cholesterol homeostasis causes hyperlipidemia and thus arteriosclerosis.

Cholesterol is an important component of a living body, is actively synthesized in the liver, and is transported through the bloodstream by carrier lipoproteins. Lipoproteins are classified into low-density lipoprotein (LDL) and high-density lipoprotein (HDL) based on their densities. LDL is responsible for transporting cholesterol from the liver to peripheral cells. LDL becomes oxidized LDL due to active oxygen in a living body, oxidized LDL is phagocytosed by macrophages, and foam macrophages accumulate and thicken the arteries, thus forming an arteriosclerotic plaque. Excessive cholesterol in arteriosclerotic plaque macrophages is transported to the liver by HDL (reverse cholesterol transport). Reverse cholesterol transport is composed of multiple steps, starting with cholesterol efflux by HDL. Specifically, excessive cholesterol in peripheral cells is exported by ABC transporters (such as ABCA1 and ABCG1) and effluxed by HDL. HDL matures while incorporating cholesterol, delivers cholesterol to LDL via a transfer protein called CETP, and transports cholesterol to the liver via an LDL receptor. Some directly deliver cholesterol to the liver via an HDL receptor (SR-BI).

As described above, accumulation of cholesterol in peripheral cells, including macrophages, is a cause of arteriosclerosis. On the other hand, reverse cholesterol transport from peripheral cells to the liver is one of the anti-arteriosclerotic mechanisms present in a living body. ABC transporters (such as ABCA1 and ABCG1) play an important role in reverse cholesterol transport.

Hardening of the arteries is a condition in which cholesterol, neutral fat, and the like accumulate in the arteries to thicken and harden the arteries, and various pathological conditions resulting therefrom are referred to as arteriosclerosis. Arteriosclerosis is thought to be caused by risk factors such as dyslipidemia, diabetes, hypertension, and smoking, and is known to eventually restrict or block the flow of arterial blood, thus resulting in cardiovascular diseases, cerebrovascular diseases, and the like.

The mechanism of arteriosclerosis is thought to be as follows. The lumen of a blood vessel is covered with a layer of endothelial cells, which are surrounded by tunica media smooth muscle cells to regulate the blood pressure and blood flow. When vascular endothelial cells are damaged, inflammatory cells and the like infiltrate into the vascular wall. That is, LDL in the blood enters the intima and is oxidized to become oxidized LDL, and in order to process oxidized LDL, monocytes, which are a type of white blood cells, also enter the intima and become macrophages. Macrophages incorporate oxidized LDL, become foam cells, and release various cytokines to induce chronic inflammatory reactions in localized blood vessels. This triggers transformation of tunica media smooth muscle cells, which then migrate to the intima and increase.

When cholesterol accumulates in peripheral cells including macrophages, inflammatory cytokines are produced, resulting in inflammation. In an inflammatory state, cholesterol excretion becomes slower, leading to further cholesterol accumulation and exacerbating inflammation. Such a negative spiral is a cause of arteriosclerosis. Promoting cholesterol excretion in peripheral cells including macrophages helps in inhibiting inflammation and thus preventing arteriosclerosis.

Accordingly, it is presumed that arteriosclerosis can be prevented by achieving promotion of cholesterol excretion through promoting ABC transporter expression or irrespectively of promoting ABC transporter expression (i.e., independently of promoting ABC transporter expression). Further, it is presumed that arteriosclerosis can be prevented by achieving promotion of cholesterol excretion through promoting ABC transporter expression or irrespectively of promoting ABC transporter expression (i.e., independently of promoting ABC transporter expression) and achieving inhibition of inflammation through promoting cholesterol excretion. Furthermore, it is presumed that arteriosclerosis can be prevented by achieving inhibition of inflammation through inhibiting inflammatory cytokine production.

Thus, an object of the present invention is to provide an ABC transporter expression promoting agent, a cholesterol excretion promoting agent, an inflammatory cytokine production inhibiting agent, an anti-inflammatory agent, and an arteriosclerosis preventing agent.

### Solution to Problem

The present invention provides the following agents.
[A1] An ABC transporter expression promoting agent, containing at least one selected from 10-hydroxypalmitic acid and 16-hydroxypalmitic acid.
[A2] The ABC transporter expression promoting agent according to [A1], wherein the ABC transporter expression promoting agent contains a Camembert cheese extract, and wherein the Camembert cheese extract contains at least one selected from 10-hydroxypalmitic acid and 16-hydroxypalmitic acid.
[A3] A cholesterol excretion promoting agent, containing at least one selected from 10-hydroxypalmitic acid and 16-hydroxypalmitic acid.
[A4] The cholesterol excretion promoting agent according to [A3], wherein the cholesterol excretion promoting agent contains a Camembert cheese extract, and wherein the Camembert cheese extract contains at least one selected from 10-hydroxypalmitic acid and 16-hydroxypalmitic acid.
[A5] The cholesterol excretion promoting agent according to [A3] or [A4], wherein the cholesterol excretion promoting agent is administered to a subject in an inflammatory state.
[A6] An inflammatory cytokine production inhibiting agent, containing at least one selected from 10-hydroxypalmitic acid and 16-hydroxypalmitic acid.
[A7] The inflammatory cytokine production inhibiting agent according to [A6], wherein the inflammatory cytokine production inhibiting agent contains a Camembert cheese extract, and wherein the Camembert cheese extract contains at least one selected from 10-hydroxypalmitic acid and 16-hydroxypalmitic acid.
[A8] An anti-inflammatory agent, containing at least one selected from 10-hydroxypalmitic acid and 16-hydroxypalmitic acid.
[A9] The anti-inflammatory agent according to [A8], wherein the anti-inflammatory agent contains a Camembert cheese extract, and wherein the Camembert cheese extract contains at least one selected from 10-hydroxypalmitic acid and 16-hydroxypalmitic acid.
[A10] An arteriosclerosis preventing agent, containing at least one selected from 10-hydroxypalmitic acid and 16-hydroxypalmitic acid.
[A11] The arteriosclerosis preventing agent according to [A10], wherein the arteriosclerosis preventing agent contains a Camembert cheese extract, and wherein the Camembert cheese extract contains at least one selected from 10-hydroxypalmitic acid and 16-hydroxypalmitic acid.

The present invention also provides the following use.
[B1] Use of at least one selected from 10-hydroxypalmitic acid and 16-hydroxypalmitic acid in the manufacture of an ABC transporter expression promoting agent.
[B2] Use of a Camembert cheese extract in the manufacture of an ABC transporter expression promoting agent, wherein the Camembert cheese extract contains at least one selected from 10-hydroxypalmitic acid and 16-hydroxypalmitic acid.
[B3] Use of at least one selected from 10-hydroxypalmitic acid and 16-hydroxypalmitic acid in the manufacture of a cholesterol excretion promoting agent.
[B4] Use of a Camembert cheese extract in the manufacture of a cholesterol excretion promoting agent, wherein the Camembert cheese extract contains at least one selected from 10-hydroxypalmitic acid and 16-hydroxypalmitic acid.
[B5] The use according to [B3] or [B4], wherein the cholesterol excretion promoting agent is administered to a subject in an inflammatory state.
[B6] Use of at least one selected from 10-hydroxypalmitic acid and 16-hydroxypalmitic acid in the manufacture of an inflammatory cytokine production inhibiting agent.
[B7] Use of a Camembert cheese extract in the manufacture of an inflammatory cytokine production inhibiting agent, wherein the Camembert cheese extract contains at least one selected from 10-hydroxypalmitic acid and 16-hydroxypalmitic acid.
[B8] Use of at least one selected from 10-hydroxypalmitic acid and 16-hydroxypalmitic acid in the manufacture of an anti-inflammatory agent.
[B9] Use of a Camembert cheese extract in the manufacture of an anti-inflammatory agent, wherein the Camembert cheese extract contains at least one selected from 10-hydroxypalmitic acid and 16-hydroxypalmitic acid.
[B10] Use of at least one selected from 10-hydroxypalmitic acid and 16-hydroxypalmitic acid in the manufacture of an arteriosclerosis preventing agent.
[B11] Use of a Camembert cheese extract in the manufacture of an arteriosclerosis preventing agent, wherein the Camembert cheese extract contains at least one selected from 10-hydroxypalmitic acid and 16-hydroxypalmitic acid.

The present invention also provides the following methods.
[C1] A method for promoting ABC transporter expression, the method including a step of administering at least one selected from 10-hydroxypalmitic acid and 16-hydroxypalmitic acid to a subject in need of promotion of ABC transporter expression.
[C2] A method for promoting ABC transporter expression, the method including a step of administering a Camembert cheese extract to a subject in need of promotion of ABC transporter expression, wherein the Camembert cheese extract contains at least one selected from 10-hydroxypalmitic acid and 16-hydroxypalmitic acid.
[C3] A method for promoting cholesterol excretion, the method including a step of administering at least one selected from 10-hydroxypalmitic acid and 16-hydroxypalmitic acid to a subject in need of promotion of cholesterol excretion.
[C4] A method for promoting cholesterol excretion, the method including a step of administering a Camembert cheese extract to a subject in need of promotion of cholesterol excretion, wherein the Camembert cheese extract contains at least one acid selected from 10-hydroxypalmitic acid and 16-hydroxypalmitic acid.
[C5] The method according to [C3] or [C4], wherein at least one selected from 10-hydroxypalmitic acid and 16-hydroxypalmitic acid or the Camembert cheese extract is administered to a subject in an inflammatory state.
[C6] A method for inhibiting inflammatory cytokine production, the method including a step of administering at least one selected from 10-hydroxypalmitic acid and 16-hydroxypalmitic acid to a subject in need of inhibition of inflammatory cytokine production.
[C7] A method for inhibiting inflammatory cytokine production, the method including a step of administering a Camembert cheese extract to a subject in need of inhibition of inflammatory cytokine production, wherein the Camembert cheese extract contains at least one acid selected from 10-hydroxypalmitic acid and 16-hydroxypalmitic acid.
[C8] A method for preventing or treating inflammation, the method including a step of administering at least one selected from 10-hydroxypalmitic acid and 16-hydroxypalmitic acid to a subject in need of prevention or treatment of inflammation.
[C9] A method for preventing or treating inflammation, the method including a step of administering a Camembert cheese extract to a subject in need of prevention or treatment of inflammation, wherein the Camembert cheese extract contains at least one selected from 10-hydroxypalmitic acid and 16-hydroxypalmitic acid.
[C10] A method for preventing arteriosclerosis, the method including a step of administering at least one selected from 10-hydroxypalmitic acid and 16-hydroxypalmitic acid to a subject in need of prevention of arteriosclerosis.
[C11] A method for preventing arteriosclerosis, the method including a step of administering a Camembert cheese extract to a subject in need of prevention of arteriosclerosis, wherein the Camembert cheese extract contains at least one acid selected from 10-hydroxypalmitic acid and 16-hydroxypalmitic acid.

The present invention also provides the following use.
[D1] Use of at least one selected from 10-hydroxypalmitic acid and 16-hydroxypalmitic acid as an active ingredient of an ABC transporter expression promoting agent.
[D2] Use of a Camembert cheese extract as an active ingredient of an ABC transporter expression promoting agent, wherein the Camembert cheese extract contains at least one selected from 10-hydroxypalmitic acid and 16-hydroxypalmitic acid.
[D3] Use of at least one selected from 10-hydroxypalmitic acid and 16-hydroxypalmitic acid as an active ingredient of a cholesterol excretion promoting agent.
[D4] Use of a Camembert cheese extract as an active ingredient of a cholesterol excretion promoting agent, wherein the Camembert cheese extract contains at least one selected from 10-hydroxypalmitic acid and 16-hydroxypalmitic acid.
[D5] The use according to [D3] or [D4], wherein the cholesterol excretion promoting agent is administered to a subject in an inflammatory state.
[D6] Use of at least one selected from 10-hydroxypalmitic acid and 16-hydroxypalmitic acid as an active ingredient of an inflammatory cytokine production inhibiting agent.
[D7] Use of a Camembert cheese extract as an active ingredient of an inflammatory cytokine production inhibiting agent, wherein the Camembert cheese extract contains at least one selected from 10-hydroxypalmitic acid and 16-hydroxypalmitic acid.
[D8] Use of at least one selected from 10-hydroxypalmitic acid and 16-hydroxypalmitic acid as an active ingredient of an anti-inflammatory agent.
[D9] Use of a Camembert cheese extract as an active ingredient of an anti-inflammatory agent, wherein the Camembert cheese extract contains at least one selected from 10-hydroxypalmitic acid and 16-hydroxypalmitic acid.
[D10] Use of at least one selected from 10-hydroxypalmitic acid and 16-hydroxypalmitic acid as an active ingredient of an arteriosclerosis preventing agent.
[D11] Use of a Camembert cheese extract as an active ingredient of an arteriosclerosis preventing agent, wherein the Camembert cheese extract contains at least one selected from 10-hydroxypalmitic acid and 16-hydroxypalmitic acid.

### Effects of Invention

The present invention provides an ABC transporter expression promoting agent, a cholesterol excretion promoting agent, an inflammatory cytokine production inhibiting agent, an anti-inflammatory agent, and an arteriosclerosis preventing agent.

### BRIEF DESCRIPTION OF DRAWINGS

FIG. 1 shows the results of fractioning the lower-layer extract by thin layer chromatography (TLC) in Example 2.

FIG. 2 shows the results of Western blotting in Example 7.

### DESCRIPTION OF EMBODIMENTS

Hereinafter, the present invention will now be described.

The present invention provides an ABC transporter expression promoting agent (hereinafter referred to as a “first agent”), a cholesterol excretion promoting agent (hereinafter referred to as a “second agent”), an inflammatory cytokine production inhibiting agent (hereinafter referred to as a “third agent”), an anti-inflammatory agent (hereinafter referred to as a “fourth agent”), and an arteriosclerosis preventing agent (hereinafter referred to as a “fifth agent”).

### <Active Ingredient>

The first to fifth agents each contain at least one selected from 10-hydroxypalmitic acid and 16-hydroxypalmitic acid. Hereinafter, the at least one selected from 10-hydroxypalmitic acid and 16-hydroxypalmitic acid is referred to as an “active ingredient”.

In one embodiment, the first to fifth agents each contain either 10-hydroxypalmitic acid or 16-hydroxypalmitic acid.

In another embodiment, the first to fifth agents each contain both 10-hydroxypalmitic acid and 16-hydroxypalmitic acid.

The amount of the active ingredient contained in each of the first to fifth agents can be suitably adjusted in consideration of the form of each agent, the number of administrations per day for each agent, the dosage per day for each agent, and the like. The first to fifth agents each may be composed solely of the active ingredient. The amount of the active ingredient contained in each of the first to fifth agents can be adjusted to, for example, 0.00000001% by mass or more and 10% by mass or less, preferably 0.0000001% by mass or more and 1% by mass or less, and more preferably 0.000001% by mass or more and 0.1% by mass or less, based on the mass of each of the first to fifth agents. In a case where the first to fifth agents each contain one of 10-hydroxypalmitic acid or 16-hydroxypalmitic acid, the amount of the active ingredient means the amount of the one compound, and in a case where the first to fifth agents each contain two of 10-hydroxypalmitic acid and 16-hydroxypalmitic acid, the amount of the active ingredient means the total amount of the two compounds.

10-Hydroxypalmitic acid may be contained in a free form in each of the first to fifth agents, or may be contained in each of the first to fifth agents in the form of being bonded to glycerin (i.e., in the form of a monoglyceride, diglyceride, or triglyceride containing 10-hydroxypalmitic acid). 16-Hydroxypalmitic acid may be contained in a free form in each of the first to fifth agents, or may be contained in each of the first to fifth agents in the form of being bonded to glycerin (i.e., in the form of a monoglyceride, diglyceride, or triglyceride containing 16-hydroxypalmitic acid). The phrase “the first to fifth agents each contain 10-hydroxypalmitic acid” encompasses a case where the first to fifth agents each contain 10-hydroxypalmitic acid in a free form and also a case where the first to fifth agents each contain 10-hydroxypalmitic acid in the form of being bonded to glycerin (i.e., in the form of a monoglyceride, diglyceride, or triglyceride containing 10-hydroxypalmitic acid). The phrase “the first to fifth agents each contain 16-hydroxypalmitic acid” encompasses a case where the first to fifth agents each contain 16-hydroxypalmitic acid in a free form and also a case where the first to fifth agents each contain 16-hydroxypalmitic acid in the form of being bonded to glycerin (i.e., in the form of a monoglyceride, diglyceride, or triglyceride containing 16-hydroxypalmitic acid).

10-Hydroxypalmitic acid may be contained in the form of a pharmaceutically acceptable salt or solvate thereof in each of the first to fifth agents. 16-Hydroxypalmitic acid may be contained in the form of a pharmaceutically acceptable salt or solvate thereof in each of the first to fifth agents. The expression that “the first to fifth agents each contain 10-hydroxypalmitic acid” encompasses a case where the first to fifth agents each contain 10-hydroxypalmitic acid in the form of a pharmaceutically acceptable salt or solvate thereof. The expression that “the first to fifth agents each contain 16-hydroxypalmitic acid” encompasses a case where the first to fifth agents each contain 16-hydroxypalmitic acid in the form of a pharmaceutically acceptable salt or solvate thereof. Examples of the pharmaceutically acceptable salt include an acid addition salt, a metal salt, an ammonium salt, and an organic amine addition salt. Examples of the pharmaceutically acceptable acid addition salt include inorganic acid salts such as hydrochloride, hydrobromide, nitrate, sulfate, and phosphate; and organic acid salts such as acetate, oxalate, maleate, fumarate, citrate, benzoate, and methanesulfonate. Examples of the pharmaceutically acceptable metal salt include alkali metal salts such as sodium salts and potassium salts, alkaline earth metal salts such as magnesium salts and calcium salts, aluminum salts, and zinc salts. Examples of the pharmaceutically acceptable ammonium salt include ammonium, tetramethylammonium, and the like. Examples of the pharmaceutically acceptable organic amine addition salt include addition salts of morpholine, piperidine, and the like.

10-Hydroxypalmitic acid and 16-hydroxypalmitic acid can be contained in Camembert cheese. Accordingly, the first to fifth agents may each contain a Camembert cheese extract, and the Camembert cheese extract may contain at least one selected from 10-hydroxypalmitic acid and 16-hydroxypalmitic acid. The safety of 10-hydroxypalmitic acid and 16-hydroxypalmitic acid is supported by extensive human dietary experience, as exemplified by Camembert cheese.

The Camembert cheese extract can be obtained by subjecting Camembert cheese, which is an extraction raw material, to an extraction treatment using an extraction solvent. Before the extraction treatment, the extraction raw material may be subjected as necessary to a drying treatment and/or a pulverization treatment. Also, before the extraction treatment, the extraction raw material may be subjected as necessary to a degreasing treatment using a non-polar solvent (for example, hexane).

The extraction treatment is a treatment for extracting the target component (10-hydroxypalmitic acid and/or 16-hydroxypalmitic acid) from the extraction raw material using an extraction solvent, and can be carried out according to a conventional method. The extraction treatment can be carried out by, for example, bringing the extraction raw material into contact with an extraction solvent. The amount of the extraction solvent used is, for example, 0.1 to 10 L, preferably 0.25 to 5 L, and more preferably 0.5 to 2.5 L per 100 g of the extraction raw material. The temperature at which the extraction raw material is brought into contact with the extraction solvent is, for example, 0 to 40° C., preferably 15 to 40° C., and more preferably 25 to 40° C. The time for which the extraction raw material is brought into contact with the extraction solvent is, for example, 0.01 to 96 hours, preferably 0.1 to 48 hours, and more preferably 0.5 to 24 hours. The extraction raw material can be brought into contact with the extraction solvent by, for example, immersing the extraction raw material in the extraction solvent. After the extraction treatment, the mixture of the extraction raw material and the extraction solvent is subjected to a solid-liquid separation treatment to remove the extraction residue, and thus an extracted solution can be obtained. The solid-liquid separation treatment can be selected from, for example, filtration, centrifugation, decantation, and the like. The extracted solution can be diluted or concentrated to give a diluted solution or a concentrated solution. The extracted solution, the diluted solution, or the concentrated solution can be dried to give a dried material. The extracted solution, the diluted solution, the concentrated solution, or the dried material can be purified to give a crude purified material or a purified material. Dilution, concentration, drying, and purification can each be carried out according to conventional methods. The extract encompasses any form of the extracted solution, diluted solution, concentrated solution, dried material, crude purified material, and purified material.

The extraction solvent is not particularly limited as long as the target component (10-hydroxypalmitic acid and/or 16-hydroxypalmitic acid) can be extracted. One solvent may be used as an extraction solvent, or a mixed solvent of two or more solvents may be used as an extraction solvent. The extraction solvent is preferably used at room temperature or at a temperature below the boiling point.

The extraction solvent may be a polar solvent or a non-polar solvent as long as the target component (10-hydroxypalmitic acid and/or 16-hydroxypalmitic acid) can be extracted. When the target component (10-hydroxypalmitic acid and/or 16-hydroxypalmitic acid) is in the form of a free fatty acid, a polar solvent such as a low-polarity solvent can be used as an extraction solvent. When the target component (10-hydroxypalmitic acid and/or 16-hydroxypalmitic acid) is in the form of being bonded to glycerin (i.e., in the form of a monoglyceride, diglyceride, or triglyceride containing 10-hydroxypalmitic acid and/or 16-hydroxypalmitic acid), a non-polar solvent can be used as an extraction solvent. The extraction solvent can be selected from, for example, acetone, ethanol, ethyl methyl ketone, glycerin, ethyl acetate, methyl acetate, diethyl ether, cyclohexane, dichloromethane, edible fat and oil, 1,1,2-tetrafluoroethane, 1,1,2-trichloroethene, carbon dioxide, 1-butanol, 2-butanol, butane, 1-propanol, 2-propanol, propane, propylene glycol, hexane, water, methanol, acetonitrile, and the like. From the viewpoint of efficiently extracting the target component (10-hydroxypalmitic acid and/or 16-hydroxypalmitic acid), the extraction solvent preferably contains acetonitrile.

In one embodiment, hexane and acetonitrile can be used as extraction solvents. A mixed solvent of hexane and acetonitrile may be added to the extraction raw material, or acetonitrile and hexane may be sequentially added to the extraction raw material and mixed. The volume ratio of hexane and acetonitrile (volume of hexane:volume of acetonitrile) is, for example, 1:10 to 10:1, preferably 1:5 to 5:1, and more preferably 1:2 to 2:1. When hexane and acetonitrile are used as extraction solvents, the acetonitrile layer (the lower layer) can be obtained as an extracted solution containing at least one selected from 10-hydroxypalmitic acid and 16-hydroxypalmitic acid. The resulting extracted solution can be subjected to a treatment such as dilution, concentration, drying, purification, or the like according to a conventional method to give a diluted solution, a concentrated solution, a dried material, a crude purified material, a purified material, or the like.

### <Applications of First Agent>

10-Hydroxypalmitic acid and 16-hydroxypalmitic acid each have an ABC transporter expression promoting activity. Accordingly, the first agent can be administered to a subject to promote ABC transporter expression. The subject is preferably a mammal such as a human, mouse, rat, hamster, rabbit, dog, cat, pig, cattle, horse, or monkey, and is more preferably a human. Promotion of ABC transporter expression encompasses promotion of ABC transporter gene expression and promotion of ABC transporter (protein) expression. Promotion of ABC transporter gene expression is preferably promotion of mRNA expression, more preferably promotion of mRNA transcription. Promotion of ABC transporter (protein) expression is preferably achieved through promotion of gene expression.

In one embodiment, the first agent can be administered to a subject to promote expression of an ABC transporter gene (preferably promote mRNA expression, and more preferably promote mRNA transcription). Promotion of ABC transporter gene expression can be confirmed by an increase in mRNA level after administration of the first agent, compared to the mRNA level before administration of the first agent. The mRNA level after administration of the first agent is preferably at least 1.5 times higher, more preferably at least 2 times higher, and even more preferably at least 2.5 times higher than the mRNA level before administration of the first agent. The mRNA level can be measured according to a conventional method such as RT-PCR. Promotion of ABC transporter gene expression can be confirmed by an increase in ABC transporter protein expression level after administration of the first agent, compared to the ABC transporter protein expression level before administration of the first agent. The ABC transporter protein expression level after administration of the first agent is preferably at least 1.25 times higher, more preferably at least 1.5 times higher, and even more preferably at least 2.0 times higher than the ABC transporter protein expression level before administration of the first agent. The protein expression level can be measured according to a conventional method such as Western blotting.

In another embodiment, the first agent can be administered to a subject to promote ABC transporter expression. Promotion of ABC transporter expression can be confirmed by an increase in ABC transporter protein expression level after administration of the first agent, compared to the ABC transporter protein expression level before administration of the first agent. The ABC transporter protein expression level after administration of the first agent is preferably at least 1.25 times higher, more preferably at least 1.5 times higher, and even more preferably at least 2.0 times higher than the ABC transporter protein expression level before administration of the first agent. The protein expression level can be measured according to a conventional method such as Western blotting.

Examples of the ABC transporter include ABCA subfamily (such as ABCA1 and ABCA3), ABCB subfamily (such as ABCB1, ABCB4, and ABCB11), ABCC subfamily (such as ABCC1, ABCC2, ABCC7, ABCC8, and ABCC9), ABCD subfamily (such as ABCD1), and ABCG subfamily (such as ABCG1, ABCG2, ABCG4, and ABCG5/8). Among these, ABCA1 and ABCG1 are preferred. That is, the first agent is preferably administered to a subject to promote expression of at least one selected from ABCA1 and ABCG1.

Cells that express the ABC transporter is not particularly limited, and suitable specific examples include macrophages, astroglial cells, nerve cells, pancreatic beta cells, adipocytes, and fibroblasts. That is, the first agent is preferably administered to a subject to promote ABC transporter expression in cells selected from macrophages, astroglial cells, nerve cells, pancreatic beta cells, adipocytes, and fibroblasts. Cells are preferably derived from a mammal such as a human, mouse, rat, hamster, rabbit, dog, cat, pig, cattle, horse, or monkey, and more preferably derived from a human. Cells may be cells in a living body or isolated cells. Isolated cells can be maintained and cultured according to conventional methods.

### <Applications of Second Agent>

10-Hydroxypalmitic acid and 16-hydroxypalmitic acid each have a cholesterol excretion promoting activity. Accordingly, the second agent can be administered to a subject to promote cholesterol excretion. The cholesterol excretion promoting activity can be exhibited through, but is not particularly limited to, an ABC transporter expression promoting activity (in particular, the activity for promoting expression of at least one selected from ABCA1 and ABCG1). The cholesterol excretion promoting activity of each of 10-hydroxypalmitic acid and 16-hydroxypalmitic acid can be exhibited irrespectively of the ABC transporter expression promoting activity (i.e., independently of the ABC transporter expression promoting activity). For example, 10-hydroxypalmitic acid and 16-hydroxypalmitic acid can each further promote cholesterol excretion even in a state where ABC transporter expression has already been induced (i.e., even in a state where cholesterol excretion has already been promoted through the ABC transporter expression promoting activity). The subject is preferably a mammal such as a human, mouse, rat, hamster, rabbit, dog, cat, pig, cattle, horse, or monkey, and is more preferably a human. Promotion of cholesterol excretion encompasses promotion of excretion of cholesterol in cells to the outside of the cells and promotion of excretion of cholesterol in the body to the outside of the body. For example, ABCG5/8 is thought to be involved in cholesterol excretion from the liver into bile. Also, ABCA1 and ABCG1 can indirectly contribute to the excretion of cholesterol in the body to the outside of the body by activating the reverse cholesterol transport system from peripheral cells to the liver. Promotion of excretion of cholesterol in the body to the outside of the body is preferably achieved through promotion of excretion of cholesterol in cells to the outside of the cells. Cholesterol encompasses free cholesterol and a cholesterol ester (cholesterol esterified with a fatty acid). Cholesterol also encompasses (3β)-cholest-5-en-3-ol, cholest-5-en-3β-ol, and derivatives and analogs thereof, as described in International Publication No. WO 2013/061969.

In one embodiment, the second agent can be administered to a subject to promote excretion of cholesterol in cells to the outside of cells. Cells in which cholesterol accumulates are not particularly limited, and examples include macrophages, adipocytes, and vascular endothelial cells. Among these, macrophages, adipocytes, and the like are preferred. That is, the second agent is preferably administered to a subject to promote excretion of cholesterol in cells selected from macrophages and adipocytes to the outside of the cells. Cells are preferably derived from a mammal such as a human, mouse, rat, hamster, rabbit, dog, cat, pig, cattle, horse, or monkey, and more preferably derived from a human. Cells may be cells in a living body or isolated cells. Isolated cells can be maintained and cultured according to conventional methods.

In another embodiment, the second agent can be administered to a subject to promote excretion of cholesterol in tissue to the outside of the tissue. Examples of the tissue in which cholesterol accumulates include, but are not limited to, central tissue and peripheral tissue. That is, the second agent can be administered to a subject to promote excretion of cholesterol in tissue selected from central tissue and peripheral tissue to the outside of the tissue. Tissue is preferably derived from a mammal such as a human, mouse, rat, hamster, rabbit, dog, cat, pig, cattle, horse, or monkey, and more preferably derived from a human. Tissue may be tissue in a living body or isolated tissue. Isolated tissue can be maintained and cultured according to conventional methods.

The second agent may be administered to a subject in an inflammatory state (i.e., a subject suffering from inflammation) or may be administered to a subject not in an inflammatory state (i.e., a subject not suffering from inflammation). The second agent can promote cholesterol excretion regardless of whether the subject is in an inflammatory state or not. Inflammation may be inflammation caused by cholesterol accumulation in peripheral cells including macrophages or inflammation due to other causes. Inflammation encompasses various reaction mechanisms caused by various stimuli, injuries, infections, and the like. Whether inflammation has been induced or not can be determined using a known inflammatory marker. Examples of the inflammatory marker include interleukin-1 (IL-1), interleukin-6 (IL-6), interleukin-8 (IL-8), interleukin-18 (IL-18), a tumor necrosis factor (TNF-α), and a C-reactive protein (CRP). For example, an increased gene expression level (such as mRNA level) of an inflammatory marker makes it possible to judge that inflammation has been induced. The description concerning inflammation in the section <Applications of fourth agent> applies to the second agent as well.

In one embodiment, the second agent can be administered to a subject to promote excretion of cholesterol in cells in an inflammatory state to the outside of the cells. Examples of cells that can be in an inflammatory state include macrophages, leucocytic cells (such as lymphocytes and plasmacytes, etc.), mast cells, and vascular endothelial cells. Among these, macrophages, adipocytes, and the like are preferred. That is, the second agent is preferably administered to a subject to promote excretion of cholesterol in cells in an inflammatory state selected from macrophages and adipocytes to the outside of the cells. Cells are preferably derived from a mammal such as a human, mouse, rat, hamster, rabbit, dog, cat, pig, cattle, horse, or monkey, and more preferably derived from a human. Cells may be cells in a living body or isolated cells. Isolated cells can be maintained and cultured according to conventional methods.

In another embodiment, the second agent can be administered to a subject to promote excretion of cholesterol in tissue in an inflammatory state to the outside of the tissue. Examples of tissue that can be in an inflammatory state include, but are not limited to, central tissue and peripheral tissue. That is, the second agent can be administered to a subject to promote excretion of cholesterol in tissue in an inflammatory state selected from central tissue and peripheral tissue to the outside of the tissue. Tissue is preferably derived from a mammal such as a human, mouse, rat, hamster, rabbit, dog, cat, pig, cattle, horse, or monkey, and more preferably derived from a human. Tissue may be tissue in a living body or isolated tissue. Isolated tissue can be maintained and cultured according to conventional methods.

### <Applications of Third Agent>

10-Hydroxypalmitic acid and 16-hydroxypalmitic acid each have an inflammatory cytokine production inhibitory activity. Accordingly, the third agent can be administered to a subject to inhibit production of an inflammatory cytokine. The subject is preferably a mammal such as a human, mouse, rat, hamster, rabbit, dog, cat, pig, cattle, horse, or monkey, and is more preferably a human. Inhibition of inflammatory cytokine production encompasses inhibition of inflammatory cytokine gene expression (preferably inhibition of mRNA expression). The inflammatory cytokine production inhibitory activity encompasses not only complete inhibition of inflammatory cytokine production, but also partial inhibition of inflammatory cytokine production, such as inhibition of excessive inflammatory cytokine production.

In one embodiment, the third agent can be administered to a subject to inhibit inflammatory cytokine production through inhibition of inflammatory cytokine gene expression (preferably inhibition of mRNA expression). Inhibition of inflammatory cytokine gene expression can be confirmed by a decrease in mRNA level after administration of the third agent, compared to the mRNA level before administration of the third agent. The mRNA level after administration of the third agent is preferably at most 0.5 times, more preferably at most 0.1 times, and even more preferably at most 0.05 times the mRNA level before administration of the third agent. The mRNA level can be measured according to a conventional method such as RT-PCR.

Inflammatory cytokines are substances involved in inflammation associated with tissue damage, bacterial infection, viral infection, tumor, and the like, and are produced by lymphocytes, macrophages, monocytes, and the like. Examples of inflammatory cytokines include G-CSF, IL-1, IL-6, IL-8, IL-11, IL-17, IL-18, IFN-α, IFN-β, IFN-γ, TNF-α, TNF-β, and chemokines. Among these, IL-1 and IL-6 are preferred. That is, the third agent is preferably administered to a subject to inhibit production of at least one selected from IL-1 and IL-6. IL-1 encompasses IL-1a and IL-1B.

### <Applications of Fourth Agent>

10-Hydroxypalmitic acid and 16-hydroxypalmitic acid each have an anti-inflammatory activity. Accordingly, the fourth agent can be administered to a subject for anti-inflammation (i.e., prevention or treatment of inflammation). The anti-inflammatory activity can be exhibited through, but is not limited to, promotion of cholesterol excretion and/or an inflammatory cytokine production inhibitory activity. The subject is preferably a mammal such as a human, mouse, rat, hamster, rabbit, dog, cat, pig, cattle, horse, or monkey, and is more preferably a human. Prevention of inflammation encompasses inhibition, suppression, and retardation of the onset of inflammation. Treatment of inflammation encompasses retardation of the progress of inflammation as well as alleviation, reduction, amelioration, and curing of a symptom.

Inflammation may be inflammation caused by accumulation of cholesterol in peripheral cells including macrophages, or inflammation due to other causes. Inflammation encompasses various reaction mechanisms caused by various stimuli, injuries, infections, and the like. Whether inflammation has been induced or not can be determined using a known inflammatory marker. Examples of the inflammatory marker include prostaglandin, IL-1, IL-6, IL-8, IL-12, TNF-α, IFN-γ, bradykinin, complement system molecules, blood coagulation factors, and C-reactive proteins. For example, an increased gene expression level (such as mRNA level) of an inflammatory marker makes it possible to judge that inflammation has been induced. Moreover, whether inflammation has been induced or not can be determined based on the erythrocyte sedimentation rate (ESR), white blood cell count, morphological change in hematocytes and other cells, and the like.

Inflammation may be any inflammation that may occur in a subject. Examples of sites where inflammation may occur include blood vessels, intestines (such as small intestine and large intestine), nasal cavities, bronchi, skin, and an oral cavity.

Examples of inflammation include inflammation in inflammatory diseases. Inflammatory diseases are diseases accompanied by an inflammatory response, and examples include gout, arteriosclerosis, gastric ulcer, nephritis (such as glomerulonephritis, IgA nephropathy, and diabetic nephropathy), periodontal diseases (such as gingivitis and periodontitis), hepatitis (such as alcoholic hepatitis and non-alcoholic hepatitis), hepatic cirrhosis, asthma, bronchitis, cerebral infarction, aneurysm, delayed-type hypersensitivity, endometriosis, acute respiratory distress syndrome, renal transplant damage, acute myocardial infarction, diabetes, systemic lupus erythematosus, Crohn's disease, pneumonia, arthritis (such as chronic rheumatoid arthritis), endotoxin shock, sepsis resulting from infectious diseases, chronic ulcerative colitis, chronic bronchitis, cystitis, chronic osteomyelitis, reflux esophagitis, cholangitis, chronic cholecystitis, gastritis, chronic cervicitis, neuroinflammatory diseases, and inflammatory skin diseases. Examples of neuroinflammatory diseases include diabetic neuropathy, dementia (such as Alzheimer-type dementia), and multiple sclerosis. Examples of inflammatory skin diseases include atopic dermatitis, seborrheic dermatitis, diaper dermatitis, allergic contact dermatitis, irritant contact dermatitis (including dermatitis caused by contact with medicines and the like), unspecified contact dermatitis, exfoliative dermatitis, dermatitis caused by ingested substances, lichen simplex chronicus, prurigo, and pruritus. The inflammatory disease is preferably an inflammatory disease involving macrophages. Examples of inflammatory diseases involving macrophages include hepatitis, asthma, systemic lupus erythematosus, Crohn's disease, atopic dermatitis, arthritis, diabetic neuropathy, and dementia.

### <Applications of Fifth Agent>

10-Hydroxypalmitic acid and 16-hydroxypalmitic acid can each prevent arteriosclerosis by achieving promotion of cholesterol excretion through promoting ABC transporter expression (in particular, promoting the expression of at least one selected from ABCA1 and ABCG1) or irrespectively of promoting ABC transporter expression (i.e., independently of promotion of ABC transporter expression). Also, 10-Hydroxypalmitic acid and 16-hydroxypalmitic acid can each prevent arteriosclerosis by achieving promotion of cholesterol excretion through promoting the expression of an ABC transporter (in particular, promotion of the expression of at least one selected from ABCA1 and ABCG1) or irrespectively of promoting ABC transporter expression (i.e., independently of promoting ABC transporter expression), and achieving inhibition of inflammation through promoting cholesterol excretion. Also, 10-hydroxypalmitic acid and 16-hydroxypalmitic acid can each prevent arteriosclerosis by achieving inhibition of inflammation through inhibiting inflammatory cytokine production. Accordingly, the fifth agent can be administered to a subject to prevent arteriosclerosis. The subject is preferably a mammal such as a human, mouse, rat, hamster, rabbit, dog, cat, pig, cattle, horse, or monkey, and is more preferably a human. Examples of arteriosclerosis include atherosclerosis, medial sclerosis (Monckeberg arteriosclerosis), and arteriolar sclerosis. Atherosclerosis is the most common form of arteriosclerosis and is the cause of cardiovascular diseases including arteriosclerotic diseases. Accordingly, the fifth agent is also useful for preventing cardiovascular diseases. Cardiovascular diseases are diseases relating to the heart and blood vessels, and examples of cardiovascular diseases include coronary heart diseases such as angina pectoris and myocardial infarction; cerebrovascular diseases such as stroke, cerebral infarction, cerebral hemorrhage, and cerebral thrombosis; peripheral arterial diseases; rheumatic heart diseases; deep vein thrombosis; and pulmonary embolism.

### <Dosage and Administration>

The daily dose of each of the first to fifth agents can be suitably adjusted in consideration of the sex, age, weight, symptom, and the like of a subject. The daily dose of the active ingredient for an adult is, for example, 0.0000001 to 0.1 g, preferably 0.000001 to 0.01 g, and more preferably 0.00001 to 0.001 g.

The administration period, administration interval, and frequency of administration per day of each of the first to fifth agents can be suitably adjusted in consideration of the sex, age, weight, symptom, and the like of a subject. Each of the first to fifth agents is administered, for example, every day, every other day, or every third day for a period of one week or longer, preferably every day, every other day, or every third day for a period of two weeks or longer, and more preferably every day, every other day, or every third day for a period of four weeks or longer. The frequency of administration per day is not particularly limited, and is usually once, twice, or three times.

The administration route of each of the first to fifth agents is not particularly limited as long as the intended application can be achieved. In one embodiment, the administration route is oral administration. In another embodiment, the administration route is parenteral administration. Examples of parenteral administration include intranasal, ophthalmic, ear, transdermal, tracheobronchial, intrarectal, intraurinary, subcutaneous, intramuscular, intravenous, and such administration routes.

### <Dosage Form>

Each of the first to fifth agents is preferably a composition, and more preferably a food or beverage composition or a pharmaceutical composition.

Examples of the food or beverage composition include health foods, nutritional supplementary foods, functional foods, foods with health claims (such as foods for specified health uses, foods with nutrient function claims, and foods with functional claims), and foods for special dietary uses (such as powdered milk for pregnant women and lactating women).

Examples of the forms of the food or beverage composition include solid, powder, paste, semi-liquid, gel, and liquid forms.

The food or beverage composition is not particularly limited as long as it can contain 10-hydroxypalmitic acid and/or 16-hydroxypalmitic acid, and examples include instant foods such as instant noodles, retort foods, canned foods, microwavable foods, instant soups/miso soups, and freeze-dried foods; beverages such as soft drinks, fruit juice drinks, vegetable drinks, soy milk drinks, coffee drinks, tea drinks, powdered drinks, concentrated drinks, and alcoholic drinks; flour products such as bread, pasta, noodles, cake mixes, and bread crumbs; confections such as candies, caramels, chewing gum, chocolate, cookies, biscuits, bars, cakes, pies, snacks, crackers, Japanese sweets, mousse, and dessert sweets; condiments such as sauces, tomato-processed seasonings, flavoring seasonings, cooking mixes, liquid condiments, dressings, soups, and curry and stew bases; fats and oils such as processed fats and oils, butter, margarine, and mayonnaise; dairy products such as milk drinks, dairy drinks, fermented milk, cheese, yogurt, drinks containing lactic acid-producing bacteria, dairy drinks, cheese, ice cream, cream, modified milk powder, liquid milk, and solid milk; processed agricultural products such as canned agricultural foods, jam/marmalade, and cereal; frozen foods, liquid diet, and supplements. The food or beverage composition can be produced according to a conventional production method except for containing 10-hydroxypalmitic acid and/or 16-hydroxypalmitic acid.

Components, other than 10-hydroxypalmitic acid and/or 16-hydroxypalmitic acid, contained in the food or beverage composition are not particularly limited. Examples of components other than 10-hydroxypalmitic acid and/or 16-hydroxypalmitic acid include water, proteins, carbohydrates, lipids, vitamins, minerals, organic acids, organic bases, fruit juices, and flavorings. Examples of proteins include animal and vegetable proteins such as whole milk powder, skimmed milk powder, partially skimmed milk powder, casein, whey powder, whey protein, whey protein concentrate, whey protein isolate, α-casein, β-casein, κ-casein, β-lactoglobulin, α-lactalbumin, lactoferrin, soy protein, hen egg protein, and meat protein; hydrolysates of such proteins; and various milk-derived components such as butter, whey minerals, cream, whey, non-protein nitrogen, sialic acid, phospholipid, and lactose. Examples of carbohydrates include commonly used sugars, modified starch (such as dextrin, soluble starch, British starch, oxidized starch, starch ester, and starch ether), and dietary fiber. Examples of lipids include animal fats and oils such as lard, fish oil, and their fractionated oils, hydrogenated oils, and ester-exchanged oils; and vegetable fats and oils such as palm oil, safflower oil, corn oil, rapeseed oil, coconut oil, and their fractionated oils, hydrogenated oils, and ester-exchanged oils. Examples of vitamins include vitamin A, carotenes, vitamin B complex, vitamin C, vitamin D complex, vitamin E, vitamin K complex, vitamin P, vitamin Q, niacin, nicotinic acid, pantothenic acid, biotin, inositol, choline, and folic acid, and examples of minerals include calcium, potassium, magnesium, sodium, copper, iron, manganese, zinc, selenium, and whey minerals. Examples of organic acids include malic acid, citric acid, lactic acid, and tartaric acid. One of such components may be used alone, or two or more of such components may be used in combination.

Examples of the pharmaceutical composition include granules, powders, tablets (including sugar-coated tablets), pills, capsules, syrups, emulsions, and suspensions. Such preparations can be produced according to a conventional method using a pharmaceutically acceptable carrier. Examples of the pharmaceutically acceptable carrier include excipients, binders, diluents, additives, flavorings, buffers, thickeners, colorants, stabilizers, emulsifiers, dispersants, suspending agents, and preservatives. One pharmaceutically acceptable carrier may be used alone, or two or more pharmaceutically acceptable carriers may be used in combination. The pharmaceutical composition thus produced contains at least one selected from 10-hydroxypalmitic acid and 16-hydroxypalmitic acid as well as one or more pharmaceutically acceptable carriers. In one embodiment, the pharmaceutical composition is a pharmaceutical composition for oral administration (an oral agent).

Examples of the carrier include water, pharmaceutically acceptable organic solvents, collagen, polyvinyl alcohol, polyvinylpyrrolidone, carboxyvinyl polymers, sodium alginate, water-soluble dextran, water-soluble dextrin, sodium carboxymethyl starch, pectin, xanthan gum, gum arabic, casein, gelatin, agar, glycerin, propylene glycol, polyethylene glycol, petrolatum, paraffin, stearyl alcohol, stearic acid, human serum albumin, mannitol, sorbitol, lactose, pharmaceutically acceptable surfactants, and artificial cellular structures such as liposomes.

The third or fourth agent may be a skin cosmetic. The third or fourth agent is useful as a skin cosmetic for preventing or treating an inflammatory skin disease.

Examples of the skin cosmetic include toners, lotions, creams, milky lotions, sunscreens, cleansers, shaving agents, facial rinses, packs, cosmetic oils, body rinses, foundations, hair dressings, hair growing tonics, scalp care products, hair colors, hair washes, hair rinses, and treatments.

The skin cosmetic can be produced using 10-hydroxypalmitic acid and/or 16-hydroxypalmitic acid as well as a commonly used skin cosmetic ingredient. Examples of base components of the skin cosmetic include ethanol, polyhydric alcohols, water, hydrocarbons, higher fatty acids, higher alcohols, fats and oils, waxes, ethers, esters, and silicones. In addition, other components, such as surfactants, moisturizing components, barrier function improving components, antioxidative components, whitening components, antibacterial components, stabilizers, fragrances, colorants, and preservatives, may be used as well. The skin cosmetic can be produced by a conventional skin cosmetic production method. The skin cosmetic production method may include, for example, steps such as ingredient mixing, heating, dissolving, drying, and filling.

Each of the first to fifth agents may be packaged to facilitate administration of a single dose. A single dose may be in a single package or in multiple packages. When provided in a packaged form, the agent is preferably provided in a form that facilitates continuous consumption, such as a set containing an amount to be consumed over a specific period of time (for example, an amount to be consumed in several days). The package form is not particularly limited as long as a specific amount is provided, and examples include wrapping paper, sacks, soft bags, paper containers, cans, bottles, and capsules.

The application, dosage, administration method, and the like of each of the first to fifth agents may be displayed on the packaging, container, or package insert (such as a product description, a pamphlet, a promotion item, and a brand site). Here, the term “display” includes all sorts of display intended to inform consumers of the above application. Such display may be any display that enables the above application, dosage, administration method, and the like to be recalled or inferred, and may include all sorts of display irrespectively of the purpose of display, contents of display, displayed object, medium, and the like.

### EXAMPLES

### Example 1

First, 20 mL of acetonitrile was added to 2.0 g of Camembert cheese (“Meiji Hokkaido Tokachi Camembert Cheese” manufactured by Meiji Co., Ltd.), then 13 mL of hexane was added, and the mixture was stirred to perform an extraction treatment. The solvent of each of the separated upper layer (hexane layer) and lower layer (acetonitrile layer) was distilled off to give an upper-layer extract and a lower-layer extract. The cholesterol excretion promoting activity of each extract was evaluated.

Human macrophage-like THP-1 cells were seeded in a 96-well plate so as to be 2.0×10⁵ cells/well, and allowed to differentiate for 72 hours. The medium was then replaced with RPMI 1640 medium containing 0.02% by mass of bovine serum albumin (BSA), 5 μM of T0901317 (Merck), and 5 μM of 9-cis-retinoic acid (Cayman Chemical), and the cells were cultured for 24 hours to induce expression of ABC transporters (endogenous ABCA1 and ABCG1). The medium was then replaced with Dulbecco's Modified Eagle Medium (DMEM) containing 0.02% by mass of BSA and 6 mM of sodium taurocholate, and the lower-layer extract was added at a concentration (final concentration in the well) of 0.04 to 40 mg/ml in terms of the weight of Camembert cheese used as the extraction raw material (0.04 to 40 mg of cheese/mL), and the cells were cultured for 4 hours. The medium was then recovered, and the level of cholesterol excreted from the cells (the cholesterol level in the recovered medium) was measured by fluorometry using Amplex Red (Invitrogen) to evaluate the cholesterol excretion promoting activity of the lower-layer extract (n=4 to 5).

The results are shown in Table 1. In Table 1, the “cholesterol excretion level” is expressed as a relative value, with the value set to 1 when no lower-layer extract was added (when dimethyl sulfoxide (DMSO) used as a dilution solvent for each extract was solely added). Also, in Table 1, “*”, “*”, and “*” indicate that the cholesterol excretion level was statistically significantly higher than that obtained when no lower-layer extract was added (*: P<0.05, **: P<0.01, ***: P<0.001, all Dunnett's test).

As shown in Table 1, it was confirmed that the lower-layer extract had a concentration-dependent cholesterol excretion promoting activity.

TABLE 1

| Concentration of lower-layer | Cholesterol excretion level |
|---|---|
| extract in terms of weight of | (Average ± standard deviation) |
| Camembert cheese | (Expressed as a relative value, with |
| (mg of cheese/mL) | the value set to 1 when DMSO was added) |
| Not added (DMSO) | 1 ± 0.165 |
| 0.04 | 0.897 ± 0.075 |
| 0.08 | 1.288 ± 0.189 |
| 0.16 | 1.476 ± 0.371 |
| 0.31 | 1.604 ± 0.343* |
| 0.63 | 1.601 ± 0.351* |
| 1.25 | 1.681 ± 0.377** |
| 2.5 | 1.522 ± 0.195* |
| 5 | 1.451 ± 0.309 |
| 10 | 1.736 ± 0.196** |
| 20 | 2.297 ± 0.312*** |
| 40 | 4.076 ± 0.382*** |

### Example 2

To further narrow down the active components, the lower-layer extract was fractionated by thin layer chromatography (TLC). Specifically, the lower-layer extract was applied to a silica gel plate (silica gel 60, Sperco) and developed with hexane:ethyl acetate=70:30 to fractionate the lower-layer extract.

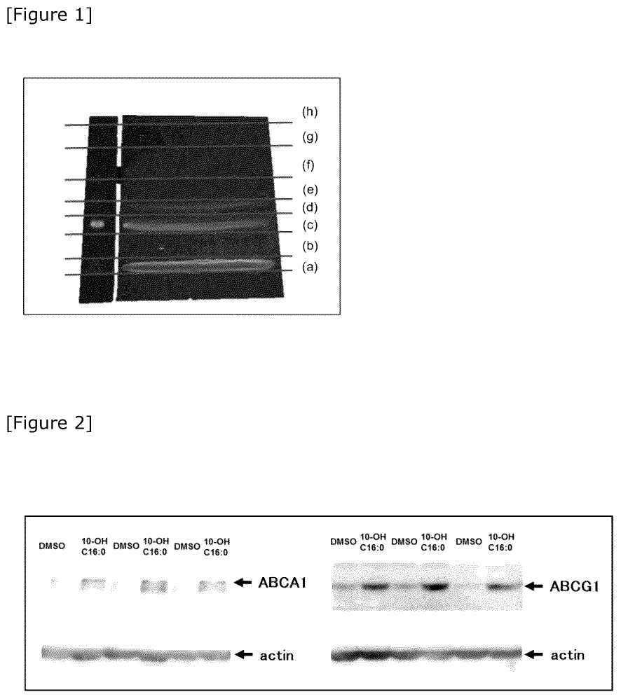

The results are shown in FIG. 1. As shown in FIG. 1, fractions (a) to (h) were obtained.

The cholesterol excretion promoting activity of each fraction was evaluated in the same manner as in Example 1 (n=1). Each fraction was added at a concentration (final concentration in the well) of 20 mg/ml in terms of the weight of Camembert cheese used as an extraction raw material (20 mg of cheese/mL).

The results are shown in Table 2A. In Table 2A, the “cholesterol excretion level” is expressed as a relative value, with the value set to 1 when no fraction was added (when DMSO used as a dilution solvent for each fraction was solely added).

As shown in Table 2A, it was confirmed that fraction (c) had a cholesterol excretion promoting activity.

TABLE 2A

| | Cholesterol excretion level |
|---|---|
| | (Expressed as a relative value, with the value set to 1 |
| Fraction | when DMSO was added) |
| (a) | 1.534 |
| (b) | 1.451 |
| (c) | 1.771 |
| (d) | 1.451 |
| (e) | 1.077 |
| (f) | 1.066 |
| (g) | 0.969 |
| (h) | 0.941 |

The concentration dependency of the cholesterol excretion promoting activity of fraction (c) was evaluated in the same manner as in Example 1 (n=3 to 4). Fraction (c) was added at a concentration (final concentration in the well) of 5 to 40 mg/ml in terms of the weight of Camembert cheese used as an extraction raw material (5 to 40 mg of cheese/mL).

The results are shown in Table 2B. In Table 2B, the “cholesterol excretion level” is expressed as a relative value, with the value set to 1 when no fraction (c) was added (when DMSO used as a dilution solvent for fraction (c) was solely added). Also, in Table 2B, “**” and “***” indicate that the cholesterol excretion level was statistically significantly higher than that obtained when no fraction (c) was added (**: P<0.01, ***: P<0.001, both Dunnett's test).

As shown in Table 2B, it was confirmed that fraction (c) had a concentration-dependent cholesterol excretion promoting activity.

TABLE 2B

| Concentration of fraction (c) | Cholesterol excretion level |
|---|---|
| in terms of weight of | (Average ± standard deviation) |
| Camembert cheese | (Expressed as a relative value, with the |
| (mg of cheese/mL) | value set to 1 when DMSO was added) |
| Not added (DMSO) | 1 ± 0.271 |
| 5 | 1.334 ± 0.047 |
| 10 | 1.398 ± 0.045 |
| 20 | 1.506 ± 0.329** |
| 40 | 1.863 ± 0.128*** |

### Example 3

The cholesterol excretion promoting activity of fraction (c) was also examined by colorimetry. Specifically, human macrophage-like THP-1 cells were seeded in a 6-well plate so as to be 4.0×10⁶ cells/well, and allowed to differentiate for 72 hours. The medium was then replaced with RPMI 1640 medium containing 0.02% by mass of bovine serum albumin (BSA), 5 μM of T0901317 (Merck), and 5 μM of 9-cis-retinoic acid (Cayman Chemical), and the cells were cultured for 24 hours to induce expression of ABC transporters (endogenous ABCA1 and ABCG1). The medium was then replaced with Dulbecco's Modified Eagle Medium (DMEM) containing 0.02% by mass of BSA and 6 mM of sodium taurocholate, fraction (c) was added at a concentration (final concentration in the well) of 20 mg/mL in terms of the weight of the Camembert cheese used as the extraction raw material (20 mg of cheese/mL), and the cells were cultured for 4 hours. The medium was then recovered, lipids were extracted using methanol and chloroform, and the level of cholesterol excreted from the cells (the cholesterol level in the recovered medium) was measured using a free cholesterol assay kit (Fujifilm Wako Pure Chemical Corporation) to evaluate the cholesterol excretion promoting activity of fraction (c) (n=3).

The results are shown in Table 3. In Table 3, the “cholesterol excretion level” is expressed as a relative value, with the value set to 1 when no fraction (c) was added (when DMSO used as a dilution solvent for fraction (c) was solely added). Also, in Table 3, “*” indicates that the cholesterol excretion level was statistically significantly higher than that obtained when no fraction (c) was added (*: P<0.05, Student's t-test).

As shown in Table 3, it was confirmed also by colorimetry that fraction (c) had a cholesterol excretion promoting activity.

TABLE 3

| Concentration of fraction (c) | Cholesterol excretion level |
|---|---|
| in terms of weight of | (Average ± standard deviation) |
| Camembert cheese | (Expressed as a relative value, with the |
| (mg of cheese/mL) | value set to 1 when DMSO was added) |
| Not added (DMSO) | 1 ± 0.080 |
| 20 | 1.193 ± 0.050* |

### Example 4

The components contained in fraction (c) were analyzed. Specifically, 0.5 mL of methanol was added to fraction (c) to dissolve the contents, the solution was allowed to pass through a 0.2 μm PTFE filter that had been washed once with methanol in advance, and the filtrate was used as a measurement sample. The measurement sample was subjected to a non-target analysis by liquid chromatography-mass spectrometry (LC-MS). The conditions of analysis by LC-MS were as follows:

### <LC>

Apparatus: Thermo Fisher Scientific Vanquish F UHPLC
Column: Accucore RT-MS 2.1×100 mm 2.6 μm (SN: 10737078)
Column temperature: 60° C.
Mobile phase A: 10 mM ammonium acetate, 0.1% acetic acid-water solution/acetonitrile (=90/10)
Mobile phase B: 2 mM ammonium acetate, 0.02% acetic acid-water/isopropyl alcohol (IPA)/acetonitrile (=2/88/10)
Flow rate: 0.25 mL/min
Injection volume: 3 μL
Gradient conditions:

TABLE 4

| Time (min) | Mobile phase A (%) | Mobile phase B (%) |
|---|---|---|
| 0 | 75 | 25 |
| 25 | 0 | 100 |
| 33 | 0 | 100 |
| 33.1 | 75 | 25 |
| 43 | 75 | 25 |

### <MS>

Equipment: Thermo Fisher Scientific™-Q Exactive™
Ionization conditions: ESI Positive (spray voltage: 3 kV), ESI Negative (spray voltage: 2 kV)
Heater temperature: 400° C.
Capillary temperature: 250° C.
Sheath gas: 50
Auxiliary Gas: 20
S-lens-level: 50
Scan conditions: Full MS/dd-MS2 (Top3)
Resolution: 70000 (Full MS), 17500 (dd-MS2)
Scan range: 100 to 1500 (Full MS), 100 to 1500 (dd-MS2)

Concerning the data obtained by LC-MS analysis, the obtained mass information was analyzed using Compound Discoverer 3.1 (manufactured by Thermo Fisher Scientific). As a result, information on active candidate components contained in the active fraction was obtained. Among them, it was found that 10-hydroxypalmitic acid, which is represented by the compositional formula C₁₆H₃₂O₃, is an active candidate component.

### Example 5A

Human macrophage-like THP-1 cells were seeded in a 96-well plate so as to be 2.0×10⁵ cells/well, and allowed to differentiate for 72 hours. The medium was then replaced with RPMI 1640 medium containing 0.02% by mass of bovine serum albumin (BSA), 10 to 40 μg/mL (final concentration in the well) of 10-hydroxypalmitic acid (Angene) was added, and the cells were cultured for 24 hours. The medium was then replaced with Dulbecco's Modified Eagle Medium (DMEM) containing 0.02% by mass of BSA and 6 mM of sodium taurocholate, and the medium was recovered after 4 hours. The level of cholesterol excreted from the cells (the cholesterol level in the recovered medium) was measured in the same manner as in Example 1 to evaluate the cholesterol excretion promoting activity of 10-hydroxypalmitic acid (n=3).

The results are shown in Table 5A. In Table 5A, the “cholesterol excretion level” is expressed as a relative value, with the value set to 1 when no 10-hydroxypalmitic acid was added (when DMSO used as a dilution solvent for 10-hydroxypalmitic acid was solely added). Also, in Table 5A, “*” and “***” indicate that the cholesterol excretion level was statistically significantly higher than that obtained when no 10-hydroxypalmitic acid was added (*: P<0.05, ***: P<0.001, both Dunnett's test).

As shown in Table 5A, it was confirmed that 10-hydroxypalmitic acid had a concentration-dependent cholesterol excretion promoting activity.

TABLE 5A

| | Cholesterol excretion level |
|---|---|
| | (Average ± standard deviation) |
| Concentration of 10- | (Expressed as a relative value, with the |
| hydroxypalmitic acid (μg/mL) | value set to 1 when DMSO was added) |
| Not added (DMSO) | 1 ± 0.045 |
| 10 | 1.553 ± 0.102 |
| 20 | 1.685 ± 0.138* |
| 30 | 2.313 ± 0.479*** |
| 40 | 1.872 ± 0.374* |

### Example 5B

The cholesterol excretion promoting activity of 10-hydroxypalmitic acid was also examined by colorimetry. Specifically, human macrophage-like THP-1 cells were seeded in a 6-well plate so as to be 4.0×10⁶ cells/well, and allowed to differentiate for 72 hours. The medium was then replaced with RPMI 1640 medium containing 0.02% by mass of bovine serum albumin (BSA), 20 μg/mL (final concentration in the well) of 10-hydroxypalmitic acid (Angene) was added, and the cells were cultured for 24 hours and then recovered. Lipids were then extracted using methanol and chloroform, and the level of cholesterol excreted from the cells (the cholesterol level in the recovered medium) was measured using a free cholesterol assay kit (Fujifilm Wako Pure Chemical Corporation) to evaluate the cholesterol excretion promoting activity of 10-hydroxypalmitic acid (n =3).

The results are shown in Table 5B. In Table 5B, the “cholesterol excretion level” is expressed as a relative value, with the value set to 1 when no 10-hydroxypalmitic acid was added (when DMSO used as a dilution solvent for 10-hydroxypalmitic acid was solely added). Also, in Table 5B, “*” indicates that the cholesterol excretion level was statistically significantly higher than that obtained when no 10-hydroxypalmitic acid was added (*: P<0.05, Student's t-test).

As shown in Table 5B, the cholesterol excretion promoting activity of 10-hydroxypalmitic acid was also confirmed by colorimetry.

TABLE 5B

| | Cholesterol excretion level |
|---|---|
| | (Average ± standard deviation) |
| Concentration of 10- | (Expressed as a relative value, with the |
| hydroxypalmitic acid (μg/mL) | value set to 1 when DMSO was added) |
| Not added (DMSO) | 1 ± 0.072 |
| 20 | 1.569 ± 0.317* |

### Example 6

Since the cholesterol excretion promoting activity of 10-hydroxypalmitic acid was confirmed, the effect of 10-hydroxypalmitic acid on ABC transporters was evaluated. Specifically, human macrophage-like THP-1 cells were seeded in a 24-well plate so as to be 1.0×10⁶ cells/well, and allowed to differentiate for 72 hours. The medium was then replaced with RPMI 1640 medium containing 0.02% by mass of bovine serum albumin (BSA), 20 μg/mL (final concentration in the well) of 10-hydroxypalmitic acid was added, the cells were cultured for 24 hours, then mRNA was extracted using RNeasy Minikit (Qiagen), reverse transcription was carried out using ReverTra Ace (Registered trademark) qPCR RT Master Mix with gDNA Remover (Toyobo), and the ABCA1 and ABCG1 mRNA expression levels were measured by real-time PCR using Thermal Cycler Dice TP800 (Takara). 18S ribosomal RNA was used as a control gene. The ABCA1 and ABCG1 mRNA expression levels were each corrected with the mRNA expression level of the control gene, and the ABC transporter expression promoting activity of 10-hydroxypalmitic acid was evaluated (n=3).

The results are shown in Tables 6A and 6B. In Tables 6A and 6B, the “mRNA expression level” is expressed as a relative value, with the value set to 1 when no 10-hydroxypalmitic acid was added (when ethanol (EtOH) used as a dilution solvent for 10-hydroxypalmitic acid was solely added). Also, in Tables 6A and 6B, “***” indicates that the ABCA1 and ABCG1 mRNA expression levels were statistically significantly higher than those obtained when no 10-hydroxypalmitic acid was added (P<0.001, Student's t-test).

As shown in Tables 6A and 6B, it was confirmed that 10-hydroxypalmitic acid had an ABC transporter expression promoting activity (in particular, an ABC transporter mRNA expression promoting activity).

TABLE 6A

| | ABCA1 mRNA expression level |
|---|---|
| | (Average ± standard deviation) |
| Concentration of 10- | (Expressed as a relative value, with the |
| hydroxypalmitic acid (μg/ml) | value set to 1 when EtOH was added) |
| Not added (EtOH) | 1 ± 0.071 |
| 20 | 2.870 ± 0.267*** |

TABLE 6B

| | ABCG1 mRNA expression level |
|---|---|
| | (Average ± standard deviation) |
| Concentration of 10- | (Expressed as a relative value, with the |
| hydroxypalmitic acid (μg/mL) | value set to 1 when EtOH was added) |
| Not added (EtOH) | 1 ± 0.139 |
| 20 | 2.953 ± 0.195*** |

### Example 7

The ABCA1 and ABCG1 protein expression levels were evaluated. Specifically, cells cultured in the same manner as in Example 6 were recovered, SDS-PAGE and blotting were carried out using the 10% minigel shown in Table 7A and, after blocking, the protein expression levels were measured by Western blotting using a primary antibody (anti-mouse monoclonal ABCA1 (KM3110) or anti-rabbit polyclonal ABCG1 (H65) (Novus)) and anti-actin (Sigma) as a loading control (n=3). The protein expression levels were quantified with CS Analyzer 4 (Biorad). The ABCA1 and ABCG1 protein expression levels were each corrected with the actin protein expression level.

The results are shown in FIG. 2 and Tables 7B and 7C. In Tables 7B and 7C, the “protein expression level” is expressed as a relative value, with the value set to 1 when no 10-hydroxypalmitic acid was added (when DMSO used as a dilution solvent for 10-hydroxypalmitic acid was solely added). Also, in Tables 7B and 7C, “*” and “**” indicate that the ABCA1 and ABCG1 protein expression levels were statistically significantly higher than those obtained when no 10-hydroxypalmitic acid was added (*: P<0.05, **: P<0.01, both Student's t-test).

As shown in Tables 7B and 7C, it was confirmed that 10-hydroxypalmitic acid had an ABC transporter expression promoting activity (an ABC transporter protein expression promoting activity).

TABLE 7A
Gel composition of Western blotting

| | Running gel | | | |
|---|---|---|---|---|
| | 10% | | Stacking gel | |
| 30% Acrylamide-0.8% bis | 2.5 | mL | 0.8 | mL |
| 1.5M Tris-Cl (pH 8.9) | 1.9 | mL | | |
| 1.1M Tris-Cl (pH 6.8) | | | 0.25 | mL |
| 0.25M EDTA (pH 7.4) | 0.25 | mL | 40 | μL |
| 10% SDS | 0.3 | mL | 53 | μL |
| H₂O | 2.65 | mL | 3.8 | mL |
| TEMED | 10 | μL | 10 | μL |
| 10% APS | 75 | μL | 50 | μL |

TABLE 7B

| | ABCA1 protein expression level |
|---|---|
| | (Average ± standard deviation) |
| Concentration of 10- | (Expressed as a relative value, with the |
| hydroxypalmitic acid (μg/mL) | value set to 1 when DMSO was added) |
| Not added (DMSO) | 1 ± 0.226 |
| 20 | 2.607 ± 0.559** |

TABLE 7C

| | ABCG1 protein expression level |
|---|---|
| | (Average ± standard deviation) |
| Concentration of 10- | (Expressed as a relative value, with the |
| hydroxypalmitic acid (μg/mL) | value set to 1 when DMSO was added) |
| Not added (DMSO) | 1 ± 0.427 |
| 20 | 2.541 ± 0.815* |

The above results indicate that 10-hydroxypalmitic acid promotes cholesterol excretion through an ABC transporter expression promoting activity.

### Example 8

In the same manner as in Example 5A, the cholesterol excretion promoting activities of 10-hydroxypalmitic acid and other hydroxypalmitic acids (2-hydroxypalmitic acid (Tokyo Chemical Industry Co., Ltd.), 3-hydroxypalmitic acid (Sigma-Aldrich), 16-hydroxypalmitic acid (Tokyo Chemical Industry Co., Ltd.), and 10-hydroxypalmitic acid), which may be contained in Camembert cheese, as well as palmitic acid to which no hydroxyl group was added (Sigma) were evaluated (n=6 to 11).

The results are shown in Table 8. In Table 8, the “cholesterol excretion level” is expressed as a relative value, with the value set to 1 when neither hydroxypalmitic acid nor palmitic acid was added (when EtOH used as a dilution solvent for hydroxypalmitic acid or palmitic acid was solely added).

As shown in Table 8, it was confirmed that 10-hydroxypalmitic acid and 16-hydroxypalmitic acid had a cholesterol excretion promoting activity.

TABLE 8

| | Cholesterol excretion level |
|---|---|
| | (Average ± standard deviation) |
| | (Expressed as a relative value, with the |
| | value set to 1 when EtOH was added) |
| Not added (EtOH) | 1 ± 0.080 |
| 2-Hydroxypalmitic acid | 0.970 ± 0.175 |
| 3-Hydroxypalmitic acid | 1.094 ± 0.271 |
| 16-Hydroxypalmitic acid | 1.406 ± 0.982 |
| 10-Hydroxypalmitic acid | 1.331 ± 0.500 |
| Palmitic acid | 0.823 ± 0.268 |

From the above results, it was confirmed that a Camembert cheese extract had a cholesterol excretion promoting activity. It was also confirmed that 10-hydroxypalmitic acid and/or 16-hydroxypalmitic acid contained in Camembert cheese exhibited a cholesterol excretion promoting activity through an ABC transporter expression promoting activity.

### Example 9

Human macrophage-like THP-1 cells were seeded in a 24-well plate so as to be 1.0×10⁶ cells/well, and allowed to differentiate for 72 hours. The medium was then replaced with RPMI 1640 medium containing: 0.02% by mass of bovine serum albumin (BSA); either dimethyl sulfoxide (DMSO) or 20 μg/mL of 10-hydroxypalmitic acid; and either 50 μg/mL of oxidized LDL (oxLDL) (Invitrogen), 50 μg/mL of acetylated LDL (acLDL) (Invitrogen), 5 μM of T0901317+5 μM of 9-cis-retinoic acid (T0+RA), or 1 μg/mL of lipopolysaccharide (LPS) (Sigma), or replaced with RPMI 1640 medium (control) containing: 0.02% by mass of bovine serum albumin (BSA); and either DMSO or 20 μg/mL 10-hydroxypalmitic acid, but not containing any of oxLDL, acLDL, T0+RA, and LPS, and the macrophage-like cells were cultured for 24 hours to induce inflammation. The medium was then replaced with Dulbecco's Modified Eagle Medium (DMEM) containing 0.02% by mass of BSA and 6 mM of sodium taurocholate, and the cells were cultured for 4 hours. The cells were then recovered, and the mRNA expression levels of IL-1 and IL-6, which are inflammatory markers (inflammatory cytokines), were measured by real-time PCR in the same manner as in Example 6 (n=4 to 6).

The results are shown in Tables 9A and 9B. In Tables 9A and 9B, the “mRNA expression level” is expressed as a relative value, with the value set to 1 when RPMI 1640 medium containing 0.02% by mass BSA and DMSO but not containing any of oxLDL, acLDL, T0+RA, and LPS was used (hereinafter referred to as a “DMSO-control”). Also, in Tables 9A and 9B, “*” and “***” indicate that the IL-1 and IL-6 mRNA expression levels were statistically significantly lower than those of the DMSO-control (*: P<0.05, ***: P<0.001, both Student's t-test).

As shown in Tables 9A and 9B, it was confirmed that 10-hydroxypalmitic acid had an inflammatory cytokine production inhibiting activity (in particular, inflammatory cytokine mRNA expression inhibition) and thus an inflammation inhibiting activity.

TABLE 9A

IL-1 mRNA expression level
(Average ± standard deviation)
(Expressed as a relative value, with the
DMSO-control set to 1)

| | DMSO | 10-Hydroxypalmitic acid |
|---|---|---|
| Control | 1 ± 0.114 | 0.200 ± 0.046*** |
| oxLDL | 4.632 ± 0.824 | 0.419 ± 0.184*** |
| acLDL | 6.007 ± 4.778 | 0.389 ± 0.067*** |
| T0 + RA | 0.280 ± 0.043 | 0.185 ± 0.059* |
| LPS | 16.149 ± 4.406 | 0.450 ± 0.390*** |

TABLE 9B

IL-6 mRNA expression level
(Average ± standard deviation)
(Expressed as a relative value, with the
DMSO-control set to 1)

| | DMSO | 10-Hydroxypalmitic acid |
|---|---|---|
| Control | 1 ± 0.227 | 0.104 ± 0.032*** |
| oxLDL | 8.659 ± 1.921 | 0.167 ± 0.029*** |
| acLDL | 9.110 ± 3.271 | 0.363 ± 0.149*** |
| T0 + RA | 0.143 ± 0.054 | 0.095 ± 0.050 |
| LPS | 6.239 ± 0.960 | 0.530 ± 0.107*** |

### Example 10

Human macrophage-like THP-1 cells were seeded in a 24-well plate so as to be 1.0×10⁶ cells/well, and allowed to differentiate for 72 hours. The medium was then replaced with RPMI 1640 medium containing: 0.02% by mass of bovine serum albumin (BSA); either dimethyl sulfoxide (DMSO) or 20 μg/mL of 10-hydroxypalmitic acid; and either 50 μg/mL of oxidized LDL (oxLDL) (Invitrogen), 50 μg/mL of acetylated LDL (acLDL) (Invitrogen), 5 μM of T0901317+5 μM of 9-cis-retinoic acid (T0+RA), or 1 μg/mL of LPS (Sigma), or replaced with RPMI 1640 medium (control) containing: 0.02% by mass of BSA; and either DMSO or 20 μg/mL 10-hydroxypalmitic acid, but not containing any of oxLDL, acLDL, T0+RA, and LPS, and the macrophage-like cells were cultured for 24 hours to induce inflammation. The medium was then replaced with Dulbecco's Modified Eagle Medium (DMEM) containing 0.02% by mass of BSA and 6 mM of sodium taurocholate, and the cells were cultured for 4 hours. The medium was then recovered, and the level of cholesterol excreted from the cells (the cholesterol level in the recovered medium) was measured by fluorometry using Amplex Red to evaluate the cholesterol excretion promoting activity of 10-hydroxypalmitic acid in an inflammation-induced state (n=4).

The results are shown in Table 10. In Table 10, the “cholesterol excretion level” is expressed as a relative value, with the value set to 1 when RPMI 1640 medium containing 0.02% by mass BSA and DMSO but not containing any of oxLDL, acLDL, T0+RA, and LPS was used (hereinafter referred to as a “DMSO-control”). Also, in Table 10, “*” and “**” indicate that the cholesterol excretion level was statistically significantly higher than that of the DMSO-control (*: P<0.05, **: P<0.01, both Student's t-test).

As shown in Table 10, it was confirmed that 10-hydroxypalmitic acid had a cholesterol excretion promoting activity also in an inflammation-induced state or in a state where the expression of ABC transporters was induced by an external factor (TO +RA).

Because 10-hydroxypalmitic acid had a cholesterol excretion promoting activity also in a state where the expression of ABC transporters was induced by an external factor (T0+RA), it was confirmed that 10-hydroxypalmitic acid exhibited not only a cholesterol excretion promoting activity through an ABC transporter expression promoting activity but also a cholesterol excretion promoting activity irrespectively of the ABC transporter expression promoting activity (i.e., independently of the ABC transporter expression promoting activity).

TABLE 10

Cholesterol excretion level
(Average # standard deviation)
(Expressed as a relative value, with the
DMSO-control set to 1)

| | DMSO | 10-Hydroxypalmitic acid |
|---|---|---|
| Control | 1 ± 0.464 | 3.588 ± 1.302** |
| oxLDL | 0.874 ± 0.353 | 2.389 ± 0.628** |
| acLDL | 0.945 ± 0.337 | 1.821 ± 0.426* |
| T0 + RA | 2.879 ± 0.811 | 33.159 ± 10.920** |
| LPS | 1.238 ± 0.417 | 9.320 ± 3.477** |

1-11. (canceled)

12. A method for promoting ABC transporter expression in a subject in need thereof, the method comprising administering to the subject at least one selected from 10-hydroxypalmitic acid and 16-hydroxypalmitic acid, or a Camembert cheese extract containing at least one selected from 10-hydroxypalmitic acid and 16-hydroxypalmitic acid.

13. A method for promoting cholesterol excretion in a subject in need thereof, the method comprising administering to the subject at least one selected from 10-hydroxypalmitic acid and 16-hydroxypalmitic acid, or a Camembert cheese extract containing at least one selected from 10-hydroxypalmitic acid and 16-hydroxypalmitic acid.

14. The method according to claim 13, wherein the subject is in an inflammatory state.

15. A method for inhibiting inflammatory cytokine production in a subject in need thereof, the method comprising administering to the subject at least one selected from 10-hydroxypalmitic acid and 16-hydroxypalmitic acid, or a Camembert cheese extract containing at least one selected from 10-hydroxypalmitic acid and 16-hydroxypalmitic acid.

16. A method for preventing or treating inflammation in a subject in need thereof, the method comprising administering to the subject at least one selected from 10-hydroxypalmitic acid and 16-hydroxypalmitic acid, or a Camembert cheese extract containing at least one selected from 10-hydroxypalmitic acid and 16-hydroxypalmitic acid.

17. A method for preventing arteriosclerosis in a subject in need thereof, the method comprising administering to the subject at least one selected from 10-hydroxypalmitic acid and 16-hydroxypalmitic acid, or a Camembert cheese extract containing at least one selected from 10-hydroxypalmitic acid and 16-hydroxypalmitic acid.
